(19) United States
(12) Patent Application Publication
(10) Pub. No.: US 2023/0293395 A1
(43) Pub. Date: Sep. 21, 2023

(54) GELLED COMPOSITION COMPRISING A SHORT-CHAIN FATTY ACID SALT

(71) Applicant: L'Oreal, Paris (FR)

(72) Inventors: Axelle BLAISE, Chevilly-Larue (FR); Geraldine LEREBOUR, Chevilly-Larue (FR); Laurence SEBILLOTTE-ARNAUD, Chevilly-Larue (FR); Sabine VRIGNAUD-BARRETEAU, Chevilly-Larue (FR); Pamela WANG, Chevilly-Larue (FR)

(21) Appl. No.: 18/010,739

(22) PCT Filed: Jun. 17, 2021
(86) PCT No.: PCT/EP2021/066494

§ 371 Date: Dec. 15, 2022

(30) Foreign Application Priority Data
Jun. 19, 2020 (FR) 2006458

(51) Int. Cl. A61K 8/04; A61K 8/88; A61K 8/73; A61K 8/36; A61Q 19/00

(52) U.S. Cl. CPC A61K 8/042; A61K 8/88; A61K 8/73; A61K 8/361; A61Q 19/007

(57) ABSTRACT

A gelled composition comprising a short-chain fatty acid salt. The composition, in particular a cosmetic composition, in particular for caring for keratin materials, in particular the skin, including between 0.5% and 4% by weight of short-chain fatty acid salt and at least 90% by weight of an aqueous phase, with respect to the total weight of the composition, gelled by a hydrophilic gelling agent chosen from (i) at least one polymer chosen from polyacrylamides and 2-acrylamido-2-methylpropanesulfonic acid polymers and copolymers. (ii) at least one polysaccharide produced by microorganisms or isolated from algae, (iii) at least cellulose or one of its derivatives, (iv) at least one carboxyvinyl polymer or (v) mixtures thereof. A cosmetic process for caring for keratin materials, in particular the skin, including at least one stage of application to said keratin materials of the composition.

The present invention relates to the cosmetics field, and in particular to the cosmetic uses for the prevention and/or care of keratin materials, and especially dry skin and/or aged skin, such as hyposeborrhoeic dry skin and/or aged skin.

### TECHNICAL FIELD

Starting from 60 or 65 years old, the skin is less supple due, inter alia, to the decrease in sebum secretion.

It is known practice to introduce active agents into cosmetic and/or dermatological compositions in order to combat the signs of ageing.

The skin is a complex matrix of tissues and ensures the mediation of numerous functions for the human body. For that, the skin has specialized cells and mechanisms, many of which are unique to this organ.

Human skin is composed of two compartments, namely an upper compartment, the epidermis, and a deep compartment, the dermis. The natural human epidermis is composed mainly of four types of cells, which are: keratinocytes (which constitute the vast majority), melanocytes, sebocytes and Langerhans cells.

Each of these cell types contributes, by virtue of its intrinsic functions, toward the essential role played in the body by the skin. In particular, the sebocytes present in the sebaceous glands of the skin are cells which synthesize an oily substance called sebum.

Sebum is a product excreted by the sebaceous glands of the skin in most mammals. The excretion mechanism is a holocrine mechanism by which sebum is deposited at the skin surface. One of the roles of sebum is to provide the skin with a hydrophobic coating through the sebaceous ducts. Furthermore, human sebum has unique characteristics compared to the other mammals. In particular, it contains a very small amount of cholesterol derivatives and a large amount of squalene. In particular, sebum is a mixture of triglycerides, wax esters, squalene, cholesterol esters, cholesterol and free fatty acid.

The importance of sebum in the homeostasis of skin tissue is known: the parts of the human body which are deficient in sebum (soles of the feet and palms of the hands) reveal a morphology and aesthetics of the skin surface which are profoundly different from those of the rest of the skin. Likewise, in the skin care field, it appears that certain aesthetic disorders (dry skin) and also major dermatological disorders (premature ageing) are attributed to an absence or insufficiency of sebum at the skin surface. An example of hyposeborrhoeic dry skin, or of skin which becomes so, is observed during skin ageing. Thus, the manifestation of xerosis linked to a sebum deficiency is very frequently observed in aged individuals, and in particular in individuals over 50 (Yamamoto, A., Serizawa, S., Ito, M. and Sato, Y., Effect of aging on sebaceous gland activity and on the fatty acid composition of wax esters, J. Invest. Dermatol., 89, 507-512 (1987)).

### PRIOR ART

In order to overcome this problem and to have available a comfortable care product having a stimulating effect on sebum production, various solutions are currently provided on the cosmetics market.

Moreover, in the patent application filed on 21 Dec. 2018 in Singapore, under number 10201811547R, it is proposed to use short-chain fatty acids (SCFAs) comprising from 3 to 8 carbon atoms, in particular propionate, butyrate and valerate, with a view to increasing the synthesis of sebum lipids and also the secretion of sebum, and consequently to preventing and/or treating dry skin and/or aged skin, in particular associated with a lack of sebrun secretion.

Thus, it is already known, from US20090022819, that the lysate of a filamentous bacterium, Vitreoscilla filiformis, can be used for the prevention and/or treatment of dry skin.

In addition, the use of a Bifidobacterium lysate for the prevention and/or treatment of dry skin is described in US20090060962.

However, in these applications, the active agent is constituted of the biomass of bacteria. One of the disadvantages of the employment of this biomass is that its incorporation in cosmetic compositions is capable of causing stabilization problems, and in particular a phase separation induced by the sedimentation of the biomass.

None of the short-chain fatty acids (SCFAs) is known in the prior art as an active ingredient which is of use for the prevention and/or the treatment of dry skin and/or aged skin. In particular, propionic acid is known, from the CTFA (Personal Care Products Council) and from Shu, M. et al. [Fermentation of Propionibacterium acnes, a commensal bacterium in the human skin microbiome, as skin probiotics against methicillin-resistant Staphylococcus aureus. PloS One 8, e55380 (2013)], as an antimicrobial active agent and its salts (propionate) as a pH corrector and have never been described as acting on lipid production and sebum secretion.

Furthermore, among the short-chain fatty acids (SCFAs) known in cosmetics, acetate (a short-chain fatty acid comprising 2 carbon atoms) has been described as a lipid production inducer (Acne and Its Therapy, Guy F. Webster and Anthony V. Rawlings, p. 262). However, acetate only contributes to lipid production and not to sebum secretion. These two functions, which are lipid production and sebum secretion, are required for efficacious treatment of hyposeborrhoeic skin.

Nevertheless, the salified forms of these short-chain fatty acids, such as sodium propionate, which are water-soluble, make their dosage formulation tricky. In particular, as regards sodium propionate, since its pKa is 4.8, its salified form in cosmetic compositions for which the pH is conventionally between 6 and 7, makes its dosage formulation tricky, in particular with regard to compatibility with gelling agents conventionally used in the cosmetic industry.

In addition, it is important to maintain the pH between 6 and 7 in order to limit the risk of the formation of acid odour due to propionic acid which is given off all the more when the pH is below 5.5.

### DISCLOSURE OF THE INVENTION

There thus remains a need to have available cosmetic solutions capable of stimulating sebum production by aged skin, in particular skin more than 60 years old, indeed even more than 65 years old, and more particularly cosmetic compositions comprising at least one short-chain fatty acid salt, in particular sodium propionate, in a dosage form suitable for its application to the skin, while observing a pH conventionally used in the cosmetic industry, namely in the vicinity of 6 to 7.

The present invention is specifically targeted at meeting this need.

### SUMMARY OF THE INVENTION

Thus, according to one of its aspects, the present invention relates to a composition, in particular a cosmetic composition, especially for caring for keratin materials, especially the skin, comprising:
between 0.5% and 4% by weight of short-chain fatty acid salt, comprising a saturated or unsaturated and straight or branched aliphatic chain containing 3 to 8 carbon atoms, preferably 3 to 5 carbon atoms, with respect to the total weight of the composition, and
at least 90% by weight of an aqueous phase, with respect to the total weight of the composition, gelled by a hydrophilic gelling agent chosen from:
(i) at least one polymer chosen from polyacrylamides and 2-acrylamido-2-methylpropanesulfonic acid polymers and copolymers,
(ii) at least one polysaccharide produced by microorganisms or isolated from algae,
(iii) at least cellulose or one of its derivatives,
(iv) at least one carboxyvinyl polymer, or
(v) one of their mixtures.

The inventors have found, surprisingly, that a short-chain fatty acid salt, in particular sodium propionate, can advantageously be formulated in a gelled composition comprising at least 90% by weight of aqueous phase, with respect to the total weight of the composition, provided that it is gelled by a gelling agent chosen from those mentioned above.

The inventors have in particular observed that, although acrylic polymers hydrophobically modified by C₁₆-C₃₀ alkyl chains are known to confer advantageous compatibility with salts, this property was not verified in the presence of a short-chain fatty acid salt, in particular in the presence of sodium propionate, as reported in the experimental part below.

The inventors have found, surprisingly, that the compositions according to the invention make it possible to effectively stabilize a short-chain fatty acid salt, in particular sodium propionate, in amounts which can vary from 0.5% to 4% by weight, with respect to the total weight of the composition, in particular which can vary from 1% to 3% by weight, optionally in combination with other active agents.

This is because, as emerges from the examples appearing below, the compositions according to the invention are stable, in particular from a rheological viewpoint.

A composition according to the invention is in particular employed for caring for the skin.

Thus, the invention also relates, according to another of its aspects, to a cosmetic process for caring for keratin materials, in particular the skin, comprising at least one stage of application, to the skin, of a composition according to the invention.

The present invention also relates to a cosmetic treatment process for preventing and/or treating dry and/or aged skin, in particular hyposeborrhoeic dry and/or aged skin, comprising at least one stage of application, to the skin, of a composition according to the invention.

Other characteristics, alternative forms and advantages of the compositions according to the invention will become more clearly apparent on reading the description and the examples which will follow.

The term “keratin materials” is understood in particular to mean the skin, the lips, in particular the skin and/or the lips, and preferably the skin.

In the context of the invention, the term “skin” denotes any skin surface of the body, preferentially facial skin and the scalp. It is anticipated that the skin involved in the present patent application be human skin.

As used herein, the term “to treat” or “treatment” refers to any action targeted at improving the comfort or the well-being of an individual. This term thus covers the reduction, relief or elimination of the symptoms of dry skin and/or of aged skin, but is limited to a cosmetic treatment.

Within the meaning of the present invention, the term “to prevent” means reducing the risk of manifestation of a phenomenon, in particular, in the context of the invention, dry skin and/or aged skin.

The term “hyposeborrhoeic” is understood to mean a lack of sebum secretion, in particular an absence of or a decrease in sebum secretion of the sebaceous glands. Conventionally, a sebum content of less than 100 μg/cm², measured in the T zone of the face by the method described in FR 2 368 708, can be regarded as characteristic of hyposeborrhoeic dry skin and of aged skin.

Within the meaning of the present invention, the term “cosmetic composition” denotes a composition suitable for application to the skin, in particular a composition which comprises a physiologically acceptable medium.

The term “physiologically acceptable medium” means a medium which is suitable for the topical administration of a composition, that is to say which is compatible (non-toxic) with the skin of the face, of the body and of the scalp.

For the purposes of the present invention, the term “short-chain fatty acid” means a carboxylic acid having an aliphatic chain comprising 3 to 8 carbon atoms, preferably a carboxylic acid having an aliphatic chain comprising 3 to 5 carbon atoms.

### DETAILED DESCRIPTION

Salts of Short-Chain Fatty Acids (SCFAs) Comprising from 3 to 8 Carbon Atoms

More particularly, the short-chain fatty acids according to the invention comprise a saturated or unsaturated and linear or branched aliphatic chain containing 3 to 8 carbon atoms, preferably 3 to 5 carbon atoms.

According to one embodiment of the invention, the short-chain fatty acid comprises a saturated and linear aliphatic chain comprising from 3 to 5 carbon atoms.

According to an even more specific embodiment, the short-chain fatty acid is chosen from propionic acid, butyric acid, valeric acid and their mixtures.

Advantageously, the short-chain fatty acids according to the invention do not contain a hydroxylated aliphatic chain; preferably, the short-chain fatty acids are not α-hydroxy acids and β-hydroxy acids, in particular are not lactic acid.

The short-chain fatty acid salts according to the invention can be any salt suitable for cosmetic use. Mention may in particular be made of the calcium salts, the sodium salts, the magnesium salts and the potassium salts, the most particularly preferred being the sodium salts.

Amino acid salts can also be used. For example, a carnitine or lysine salt of short-chain fatty acids according to the invention can be used. A person skilled in the art is able to identify various other amino acids which can also be used in the context of the present invention.

Thus, according to a specific embodiment of the invention, the short-chain fatty acid salt is chosen from a salt of propionic acid, of butyric acid, of valeric acid or of one of their mixtures, it being possible for the salt to be chosen from calcium salts, sodium salts, magnesium salts and potassium salts, and more particularly sodium salts, in particular sodium propionate.

According to a specific embodiment, the short-chain fatty acid is obtained from at least one microorganism of the species Propionibacterium acnes, preferentially from at least one microorganism of the Propionibacterium acnes ATCC 6919 strain.

In another embodiment, the short-chain fatty acids according to the invention are contained in a conditioned culture medium (or supernatant) of at least one microorganism of the species Propionibacterium acnes, preferably of at least one microorganism of the Propionibacterium acnes ATCC 6919 strain.

Mention may be made, by way of examples of short-chain fatty acid salts suitable for the invention, of the sodium propionate (Ref. P1880) and the sodium butyrate (Ref. 303410) which are sold by Sigma or the sodium propionate available under the trade name Sodium Propionate from Dr. Paul Lohmann.

A “culture supernatant”, also referred to as “conditioned culture medium”, is typically obtained by culturing the microorganism involved in a medium suitable for the survival and/or the growth of the microorganism, then by separating the medium and the microorganism in order to harvest the medium brought into contact with the microorganism. Preferably, the culturing is carried out for a time and under conditions capable of making it possible for the microorganism to release, into the medium, the active agents having the desired seborrhoeic properties, in particular the short-chain fatty acids (SCFAs) according to the invention.

The environment suitable for the survival and/or the growth of the microorganism can be constituted of any nutritive medium suitable for the survival and/or the culture of the microorganism. It generally contains a source of carbon and nitrogen, such as, for example, amino acids, sugars, proteins, fatty acids, phosphates, sulfates, minerals and growth factors and vitamins, in appropriate amounts.

For the purposes of the present patent application, the terms “conditioned culture medium” or “culture supernatant” are used without distinction to denote the entire culture supernatant obtained after culture of the microorganism in question, or any fraction or by-product of the supernatant obtained by dialysis, fractionation, phase separation, filtration chromatography, affinity chromatography, precipitation, concentration, lyophilization, and the like.

In the context of the present invention, the conditioned culture medium of at least one microorganism of the species Propionibacterium acnes according to the invention can be obtained by the process comprising the following stages:
i) culturing at least one microorganism of the species Propionibacterium acnes, such as Propionibacterium acnes ATCC 6919;
ii) separating, in particular by centrifugation, the culture supernatant from the biomass;
iii) recovering the culture supernatant; and
iv) optionally stabilizing the culture supernatant, for example by filtration.

In the context of the present invention, the term “biomass” refers to the Propionibacterium acnes cells obtained after having carried out stage i).

Preferably, the filtration is carried out with a syringe filter with a pore size of 0.45 μm.

### Sodium Propionate

According to a specific embodiment, the short-chain fatty acid salt is sodium propionate. Sodium propionate has the following chemical structure:

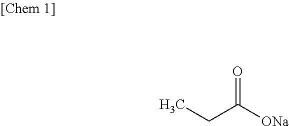

It is sometimes used as a food additive, as a preservative in particular, under the code name E281.

It is water-soluble.

### Cosmetic Composition

A composition according to the invention comprises between 0.5% and 4% by weight of short-chain fatty acid salt, with respect to the total weight of the composition, in particular between 1% and 3% by weight, more particularly still between 1.5% and 2.5% by weight.

Thus, in particular, a composition according to the invention comprises between 0.5% and 4% by weight of sodium propionate, with respect to the total weight of the composition, in particular between 1% and 3% by weight, more particularly still between 1.5% and 2.5% by weight.

In a preferred embodiment, a composition according to the invention comprises between 0.8% and 4% by weight of short-chain fatty acid, with respect to the total weight of the composition, in particular between 0.8% and 3% by weight, more particularly still between 0.8% and 2.5% by weight.

Preferably, a composition according to the invention comprises between 0.8% and 4% by weight of sodium propionate, with respect to the total weight of the composition, in particular between 0.8% and 3% by weight, even more particularly between 0.8% and 2.5% by weight.

According to a specific embodiment, a composition according to the invention comprises 2% of short-chain fatty acid salt, in particular of sodium propionate.

According to another specific embodiment, a composition according to the invention comprises 0.8% of short-chain fatty acid salt, in particular of sodium propionate.

A composition according to the invention is generally suitable for topical application to keratin materials and in particular to the skin and thus generally comprises a physiologically acceptable medium.

It is preferably a cosmetically acceptable medium, that is to say a medium which exhibits a pleasant colour, odour and feel and which does not cause any unacceptable discomfort, that is to say stinging, tautness or redness, liable to discourage the user from applying this composition.

As indicated above, the present cosmetic composition comprises at least 90°/% by weight of aqueous phase, with respect to the total weight of the composition.

According to a specific embodiment, the pH of the cosmetic composition according to the present invention is between 5.8 and 7.2, in particular between 6 and 7.

As indicated above, maintaining in such a pH range exhibits, inter alia, the advantage of limiting the risk of formation of acid odour that can be given off if propionic acid is formed, in particular for a pH of less than 5.5.

For the purpose of maintaining the pH in this value range, the composition according to the invention can comprise pH1 adjusters (acids or bases). Any pH adjuster conventionally suitable for a cosmetic application can be employed in the context of the present patent application. Typically, mention may be made of citric acid, lactic acid, tartaric acid, oxalic acid, sodium hydroxide, HEPES, sodium citrate, potassium hydroxide and triethanolamine. When the composition comprises such a pH1 adjuster, it is up to a person skilled in the art to adjust the acid or base content in order to adjust the pH to between 6 and 7 preferentially.

The pH values can also be adjusted using the various pH adjusters mentioned above, depending on the gelling agents present in the composition according to the invention.

A composition according to the present invention can be provided in the form of an aqueous gel. According to this embodiment, the composition is then devoid of fatty phase.

This aqueous gel advantageously exhibits a smooth appearance.

This aqueous gel can furthermore be more or less transparent.

Finally, according to a specific embodiment, this aqueous gel provides properties on application and to the feel of softness and of velvetiness. This property is reported in the examples by the description of “creamy” gels.

According to a specific embodiment, a composition according to the present invention can also be provided in the form of a serum. In the context of the present invention, “serum” is understood to mean a composition exhibiting a fluid, runny and slightly gelled texture.

According to another specific embodiment, the composition according to the present invention can contain up to 10% by weight of fatty phase, as described in more detail later. In this case, in the context of the present invention, such a composition can be described as “emulsified gel”.

According to a specific embodiment, the composition according to the invention is provided in the form of an aqueous gel, of a serum or of an emulsified gel.

A cosmetic composition according to the invention can typically exhibit a viscosity of between 5 poises (0.5 Pa·s) and 70 poises (7 Pa·s).

A composition according to the invention can be prepared according to techniques well known to a person skilled in the art.

### Gelling Agent

As indicated above, the composition according to the invention comprises an aqueous phase gelled by:
(i) at least one polymer chosen from polyacrylamides and 2-acrylamido-2-methylpropanesulfonic acid polymers and copolymers,
(ii) at least one polysaccharide produced by microorganisms or isolated from algae,
(iii) at least cellulose or one of its derivatives,
(iv) at least one carboxyvinyl polymer, or
(v) one of their mixtures.

The gelling agents employed in the context of the present invention are gelling agents for the aqueous phase. As such, they can be more particularly described as hydrophilic gelling agents.

Within the meaning of the present invention, “hydrophilic gelling agent” is understood to mean a compound capable of gelling the aqueous phase of the compositions according to the invention.

The gelling agent is hydrophilic and is thus present in the aqueous phase of the composition.

The gelling agent can be water-soluble or water-dispersible.

According to a specific embodiment of the invention, the aqueous phase of a composition according to the invention is gelled by 0.1% to 8% by weight of at least one hydrophilic gelling agent, with respect to the total weight of the aqueous phase.

Preferably, the aqueous phase of a composition according to the invention is gelled by 0.5% to 8% by weight of at least one hydrophilic gelling agent, more particularly still by 0.8% to 4% by weight, in particular for example by 0.8% to 3% by weight, with respect to the total weight of the aqueous phase.

According to a specific embodiment, the hydrophilic gelling agent can be chosen from (i) a polymer chosen from polyacrylamides and 2-acrylamido-2-methylpropanesulfonic acid polymers and copolymers.

Polyacrylamides and 2-acrylamido-2-methylpropanesulfonic acid Polymers and Copolymers

The polymers used which are suitable as aqueous gelling agent for the invention can be crosslinked or non-crosslinked homopolymers or copolymers comprising at least the 2-acrylamido-2-methylpropanesulfonic acid (AMPS®) monomer, in a form partially or completely neutralized by an inorganic base other than aqueous ammonia, such as sodium hydroxide or potassium hydroxide.

AMPS® (monomer) is a trademark registered in the name of Lubrizol.

They are preferably completely or virtually completely neutralized, that is to say at least 90% neutralized.

These AMPS® polymers according to the invention can be crosslinked or non-crosslinked.

When the polymers are crosslinked, the crosslinking agents can be chosen from the polyolefinically unsaturated compounds commonly used for the crosslinking of polymers obtained by radical polymerization.

Mention may be made, for example, as crosslinking agents, of divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allyl ethers of alcohols of the sugar series, or other allyl or vinyl ethers of polyfunctional alcohols, and also allyl esters of phosphoric and/or vinylphosphonic acid derivatives, or mixtures of these compounds.

According to a preferred embodiment of the invention, the crosslinking agent is chosen from methylenebisacrylamide, allyl methacrylate or trimethylolpropane triacrylate (TMPTA). The degree of crosslinking generally ranges from 0.01 mol % to 10 mol % and more particularly from 0.2 mol % to 2 mol %, with respect to the polymer.

The AMPS® polymers suitable for use in the invention are water-soluble or water-dispersible. In this case, they are either “homopolymers” comprising only AMPS® monomers and, if they are crosslinked, one or more crosslinking agents such as those defined above, or copolymers obtained from AMPS® and from one or more hydrophilic or hydrophobic ethylenically unsaturated monomers and, if they are crosslinked, one or more crosslinking agents such as those defined above. When said copolymers comprise hydrophobic ethylenically unsaturated monomers, the latter do not comprise a fatty chain and are preferably present in small amounts.

Within the meaning of the present invention, the term “fatty chain” is understood to mean any hydrocarbon chain comprising at least 7 carbon atoms.

The term “water-soluble or water-dispersible” is understood to mean polymers which, when introduced into an aqueous phase at 25° C., at a concentration by weight equal to 1%, make it possible to obtain a macroscopically homogeneous and transparent solution, that is to say a solution having a maximum light transmittance value, at a wavelength equal to 500 nm, through a sample 1 cm thick, of at least 60% and preferably of at least 70%.

The “homopolymers” according to the invention are preferably crosslinked and neutralized, and they can be obtained according to the preparation process comprising the following stages: (a) the monomer, such as AMPS®, in the free form is dispersed or dissolved in a solution of tert-butanol or of water and of tert-butanol; (b) the monomer solution or dispersion obtained in (a) is neutralized by one or more inorganic or organic bases, preferably aqueous ammonia NH₃, in an amount making it possible to obtain a degree of neutralization of the sulfonic acid functional groups of the polymer ranging from 90% to 100%; (c) the crosslinking monomer(s) is (are) added to the solution or dispersion obtained in (b); (d) a conventional radical polymerization is carried out in the presence of free radical initiators at a temperature ranging from 10° C. to 150° C., the polymer precipitating from the solution or dispersion based on tert-butanol.

The water-soluble or water-dispersible AMPS® copolymers according to the invention contain water-soluble ethylenically unsaturated monomers, hydrophobic monomers or their mixtures.

The water-soluble comonomers can be ionic or non-ionic.

Mention may be made, among the ionic water-soluble comonomers, for example, of the following compounds and their salts: (meth)acrylic acid, styrenesulfonic acid, vinylsulfonic acid and (meth)allylsulfonic acid, vinylphosphonic acid, maleic acid, itaconic acid, crotonic acid, the water-soluble vinyl monomers of following formula (A):

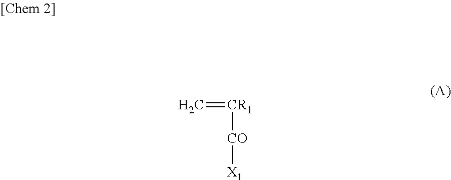

in which R₁ is chosen from H, —CH₃, —C₂H₅ or —C₃H₇; X₁ is chosen from alkyl oxides of —OR₂ type where R₂ is a saturated or unsaturated and linear or branched hydrocarbon radical having from 1 to 6 carbon atoms, substituted by at least one sulfonic (—SO₃) and/or sulfate (—SO₄⁻) and/or phosphate (—PO₄H₂) group.

Mention may be made, among the non-ionic water-soluble comonomers, for example, of (meth)acrylamide, N-vinylacetamide and N-methyl-N-vinylacetamide, N-vinylformamide and N-methyl-N-vinylformamide, maleic anhydride, vinylamine. N-vinyllactams comprising a cyclic alkyl group having from 4 to 9 carbon atoms, such as N-vinylpyrrolidone, N-butyrolactam and N-vinylcaprolactam, the vinyl alcohol of formula CH₂═CHOH, the water-soluble vinyl monomers of following formula (B):

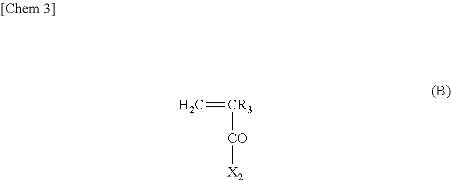

in which R₃ is chosen from H, —CH₃, —C₂H₅ or —C₃H₇; X₂ is chosen from alkyl oxides of —OR₄ type where R₄ is a saturated or unsaturated and linear or branched hydrocarbon radical having from 1 to 6 carbon atoms, optionally substituted by a halogen (iodine, bromine, chlorine or fluorine) atom; a hydroxyl (—OH) group; ether.

Mention may be made, for example, of glycidyl (meth)acrylate, hydroxyethyl methacrylate, and ethylene glycol, diethylene glycol or polyalkylene glycol (meth)acrylates.

Mention may be made, among the hydrophobic comonomers without a fatty chain, for example, of styrene and its derivatives, such as 4-butylstyrene, α-methylstyrene and vinyltoluene; vinyl acetate of formula CH₂═CH—OCOCH₃; vinyl ethers of formula CH₂═CHOR in which R is a saturated or unsaturated and linear or branched hydrocarbon radical having from 1 to 6 carbon atoms; acrylonitrile; caprolactone; vinyl chloride and vinylidene chloride; silicone derivatives resulting, after polymerization, in silicone polymers, such as methacryloyloxypropyltris(trimethylsiloxy)silane and silicone methacrylamides; the hydrophobic vinyl monomers of following formula (C):

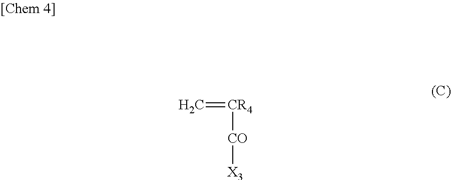

in which R₄ is chosen from H, —CH₃, —C₂H₅ or —C₃H₇; X₃ is chosen from alkyl oxides of —OR₅ type where R₅ is a saturated or unsaturated and linear or branched hydrocarbon radical having from 1 to 6 carbon atoms.

Mention may be made, for example, of methyl methacrylate, ethyl methacrylate, n-butyl (meth)acrylate, tert-butyl (meth)acrylate, cyclohexyl acrylate, isobornyl acrylate and 2-ethylhexyl acrylate.

The water-soluble or water-dispersible AMPS® polymers of the invention preferably have a molar mass ranging from 50 000 g/mol to 10 000 000 g/mol, preferably from 80 000 g/mol to 8 000 000 g/mol and more preferably still from 100 000 g/mol to 7 000 000 g/mol.

Mention may be made, as water-soluble or water-dispersible AMPS® homopolymers suitable for the invention, for example, of crosslinked or non-crosslinked polymers of sodium acrylamido-2-methylpropanesulfonate, such as that used in the commercial product Simulgel 800 (CTFA name: Sodium Polyacryloyldimethyl Taurate), crosslinked polymers of ammonium acrylamido-2-methylpropanesulfonate (INCI name: Ammonium Polyacryldimethyltauramide), such as those described in Patent EP 0 815 928 B1 and such as the product sold under the trade name Hostacerin AMPS® by Clariant.

Preferably, a composition according to the invention comprises an AMPS® homopolymer.

Mention may be made, as water-soluble or water-dispersible AMPS® copolymers in accordance with the invention, for example, of:
crosslinked acrylamide/sodium acrylamido-2-methylpropanesulfonate copolymers, such as that used in the commercial product Sepigel 305® by Seppic (CTFA name: Polyacrylamide/C₁₃-C₁₄ Isoparaffin/Laureth-7) or that used in the commercial product sold under the name Simulgel 600 (CTFA name: Acrylamide/Sodium acryloyldimethyltaurate/Isohexadecane/Polysorbate 80) by Seppic;
copolymers of AMPS® and of vinylpyrrolidone or of vinylformamide, such as that used in the commercial product sold under the name Aristoflex AVC® by Clariant (CTFA name: Ammonium Acryloyldimethyltaurate/VP Copolymer) but neutralized by sodium hydroxide or potassium hydroxide;
copolymers of AMPS® and of sodium acrylate, such as, for example, the AMPS®/sodium acrylate copolymer, such as that used in the commercial product sold under the name Simulgel EG® by Seppic or under the trade name Sepinov EM (CTFA name: Hydroxyethyl Acrylate/Sodium Acryloyldimethyltaurate Copolymer);
copolymers of AMPS® and of hydroxyethyl acrylate, such as, for example, the AMPS®/hydroxyethyl acrylate copolymer, such as that used in the commercial product sold under the name Simulgel NS® by Seppic (CTFA name: Hydroxyethyl Acrylate/Sodium Acryloyldimethyltaurate Copolymer (And) Squalane (And) Polysorbate 60), or such as the product sold under the name Sodium Acrylamido-2-Methylpropanesulfonate/Hydroxyethyl Acrylate Copolymer, such as the commercial product Sepinov EMT 10® from Seppic (INCI name: Hydroxyethyl Acrylate/Sodium Acryloyldimethyltaurate Copolymer).

Mention may be made, as preferred water-soluble or water-dispersible AMPS® homopolymers and copolymers in accordance with the invention, of crosslinked ammonium acrylamido-2-methylpropanesulfonate homopolymers (INCI name: Ammonium Polyacryldimethyltauramide), such as those described in Patent EP 0 815 928 B1 and such as the product sold under the trade name Hostacerin AMPS® by Clariant, crosslinked acrylamide/sodium acrylamido-2-methylpropanesulfonate copolymers, such as that used in the commercial product Sepigel 305® from Seppic (CTFA name: Polyacrylamide/C₁₃-C₁₄ Isoparaffin/Laureth-7), or also copolymers of AMPS® and of hydroxyethyl acrylate, such as the product sold under the name Sodium Acrylamido-2-Methylpropanesulfonate/hydroxyethyl Acrylate Copolymer, such as the commercial product Sepinov EMT 10® from Seppic (INCI name: Hydroxyethyl Acrylate/Sodium Acryloyldimethyltaurate Copolymer).

In general, a composition according to the invention can comprise from 0.1% to 8% by weight as dry matter, preferably from 0.2% to 5% by weight and more preferentially from 0.7% to 3% by weight of polyacrylamides and of 2-acrylamido-2-methylpropanesulfonic acid polymers and copolymers, with respect to the total weight of the composition.

According to a specific embodiment, the hydrophilic gelling agent can be chosen from (ii) at least one polysaccharide produced by microorganisms or isolated from algae.

In particular, the polysaccharides can be chosen from gellans, pullulan, dextrans, celluloses and their derivatives, in particular methylcelluloses, hydroxyalkylcelluloses, ethylhydroxyethylcelluloses and carboxymethylcelluloses, galactans, carrageenans, agars, gums of biopolysaccharides of microbial origin, especially scleroglucan or xanthan gums.

Advantageously, the polysaccharides can be chosen from carrageenans, in particular kappa-carrageenan, gellan gum, agar, xanthan gum, scleroglucan gum, pullulan and their mixtures. Preferably, the polysaccharide can be xanthan gum.

Such a gelling agent can be employed in a proportion of 0.1% to 8% by weight as dry matter, with respect to the total weight of the aqueous phase, in particular from 0.1% to 6% by weight, preferably between 0.5% and 2.5% by weight, especially in a proportion of approximately 1%, or also in a proportion of approximately 2%, by weight, with respect to the total weight of the aqueous phase.

More specifically, these polysaccharides suitable for the invention can be distinguished according to whether they result from microorganisms or from algae.

### Polysaccharides Produced by Microorganisms

### Xanthan

Xanthan is a heteropolysaccharide produced on an industrial scale by the aerobic fermentation of the bacterium Xanthomonas campestris. Its structure is constituted of a main chain of β(1,4)-linked β-D-glucoses, similar to cellulose. One glucose molecule in two carries a trisaccharide side chain composed of an α-D-mannose, of a β-D-glucuronic acid and of a terminal β-D-mannose. The internal mannose residue is generally acetylated on carbon 6. Approximately 30% of the terminal mannose residues carry a pyruvate group bonded in chelated form between carbons 4 and 6. The charged pyruvic acids and glucuronic acids are ionizable, and are thus responsible for the anionic nature of xanthan (negative charge down to a pH equal to 1). The content of the pyruvate and acetate residues varies according to the bacterial strain, the fermentation process, the conditions after fermentation and the purification stages. These groups may be neutralized in commercial products with Na⁺, K⁺ or Ca⁺ ions (Satia, 1986). The neutralized form can be converted into the acid form by ion exchange or by dialysis of an acidic solution.

Xanthan gums have a molecular weight of between 1 000 000 and 50 000 000 and a viscosity of between 0.6 and 1.65 Pa·s for an aqueous composition containing 1% of xanthan gum (measured at 25° C. with a Brookfield viscometer, LVT type, at 60 rpm).

Xanthan gums are represented, for example, by the products sold under the Rhodicare names by Rhodia Chimie, under the Satiaxane™ name by Cargill Texturizing Solutions (for the food, cosmetic and pharmaceutical industries), under the Novaxan™ name by ADM, under the Rhodicare CFT® trade name by PMC Ouvrie, and under the Kelzan® and Keltrol® names by CP-Kelco.

Advantageously, a composition according to the invention comprises a xanthan gum.

The xanthan gum(s) can be employed in a proportion of 0.1% to 5% by weight of dry matter with respect to the total weight of the composition, in particular from 0.5% to 3% by weight, preferably between 0.5% and 2% by weight, with respect to the total weight of the composition.

### Pullulan

Pullulan is a polysaccharide constituted of maltotriose units, which are known under the name of α(1,4)-α(1,6)-glucan. Three glucose units in maltotriose are connected by an α(1,4) glycoside bond, whereas consecutive maltotriose units are connected to each other by an α(1,6) glycoside bond.

Pullulan is produced, for example, under the reference Pullulan PF 20 by the Hayashibara group in Japan.

### Dextran and Dextran Sulfate

Dextran is a neutral polysaccharide without a charged group, is biologically inert and is prepared by fermentation of beet sugar containing solely hydroxyl groups.

It is possible to obtain dextran fractions of different molecular weights from native dextran by hydrolysis and purification. Dextran can in particular be provided in the form of dextran sulfate. Dextran is represented, for example, by the products sold under the name Dextran or Dextran T by Pharmacosmos or under the name Dextran 40 Powder or Dextran 70 Powder by Meito Sangyo Co. Dextran sulfate is sold by PK Chemical A/S under the name Dextran Sulfate.

### Succinoglycan

Succinoglycan is an extracellular polymer produced by bacterial fermentation, of high molecular weight and constituted of octasaccharide repeat units (repetition of 8 sugars). Succinoglycans are sold, for example, under the Rheozan name by Rhodia.

### Scleroglucan

Scleroglucan is a non-ionic branched homopolysaccharide constituted of β-D-glucan units. The molecules are constituted of a main linear chain formed of D-glucose units linked by β(1,3) bonds and one in three of which is linked to a D-glucose side unit by a β(1,6) bond.

A more complete description of scleroglucans and of their preparation can be found in the document U.S. Pat. No. 3,301,848.

Scleroglucan is sold, for example, under the Amigel name by Alban Muller or under the Actigum™ CS name by Cargill.

### Gellan Gum

Gellan gum is an anionic linear heteropolysaccharide based on oligosaccharide units composed of 4 monosaccharides (tetrasaccharide). D-Glucose. L-rhamnose and D-glucuronic acid in 2:1:1 proportions are present in gellan gum in the form of monomer elements.

It is sold, for example, under the name Kelcogel CG LA by CP Kelco.

Polysaccharides Isolated from Algae

### Galactans

The polysaccharide according to the invention can be a galactan in particular chosen from agar or carrageenans.

Carrageenans are anionic polysaccharides constituting the cell walls of various red algae (Rhodophyceae) belonging to the Gigartinacae, Hypneaceae, Furcellariaceae and Polyideaceae families. They are generally obtained by hot aqueous extraction from natural strains of said algae. These linear polymers, formed by disaccharide units, are composed of two D-galactopyranose units linked alternately by α(1,3) and β(1,4) bonds. They are highly sulfated (20-50%) polysaccharides and the α-D-galactopyranosyl residues can be in 3,6-anhydro form. Depending on the number and the position of ester-sulfate groups on the repeating disaccharide of the molecule, several types of carrageenans are distinguished, namely: kappa-carrageenans, which have one ester-sulfate group, iota-carrageenans, which have two ester-sulfate groups, and lambda-carrageenans, which have three ester-sulfate groups.

Carrageenans are composed essentially of salts of potassium, sodium, magnesium, triethanolamine and/or calcium and of polysaccharide ester-sulfates.

Carrageenans are sold in particular by Seppic under the Solagum® name, by Gelymar under the Carragel®, Carralact® and Carrasol® names, by Cargill under the Satiagel™ and Satiagum™ names, and by CP-Kelco under the Genulacta®, Genugel® and Genuvisco® names.

Galactans of agar type are galactose polysaccharides contained in the cell wall of some of these species of red algae (Rhodophyceae). They are formed of a polymer group, the base backbone of which is a β(1,3)-D-galactopyranose and α(1,4)-L-3,6-anhydrogalactose chain, these units repeating regularly and alternately. The differences within the agar family are due to the presence or absence of solvated methyl or carboxyethyl groups. These hybrid structures are generally present in variable percentage, depending on the species of algae and the harvesting season.

Agar is a mixture of polysaccharides (agarose and agaropectin) of high molecular weight, of between 40 000 and 300 000 g·mol⁻¹. It is obtained by manufacturing algal extraction liquors, generally by autoclaving, and by treating these liquors, which comprise approximately 2% of agar, so as to extract the latter.

Agar is produced, for example, by the B&V Agar Producers group, under the Gold Agar, Agarite and Grand Agar names by Hispanagar, and under the Agar-Agar, QSA (Quick Soluble Agar) and Puragar names by Setexam.

### Furcellaran

Furcellaran is obtained commercially from red algae Furcellaria fasztigiata. Furcellaran is produced, for example, by Est-Agar.

According to a specific embodiment, the hydrophilic gelling agent can be chosen from (iii) at least cellulose or one of its derivatives.

### Cellulose and Derivatives

Mention may be made, among cellulose derivatives, of cellulose ethers or esters (e.g.: methylcellulose, carboxymethylcellulose, hydroxymethylcellulose, hydroxyethylcellulose, hydroxypropylcellulose, hydroxymethylpropylcellulose, cellulose acetate, cellulose nitrate or nitrocellulose).

The composition according to the invention can also contain a cellulose-based associative polymer. According to the invention, the term “cellulose-based compound” is understood to mean any polysaccharide compound having, in its structure, linear sequences of anhydroglucopyranose residues (AGUs) linked together by β(1,4) glycoside bonds. The repeat unit is the cellobiose dimer. The AGUs are found in chair conformation and have 3 hydroxyl functional groups: two secondary alcohols (in positions 2 and 3) and one primary alcohol (in position 6). The polymers thus formed combine together by intermolecular bonds of hydrogen bond type, thus conferring a fibrillar structure on the cellulose (approximately 1500 molecules per fibre).

The degree of polymerization differs enormously according to the origin of the cellulose; its value can vary from a few hundred to several tens of thousands.

Cellulose exhibits the following chemical structure:

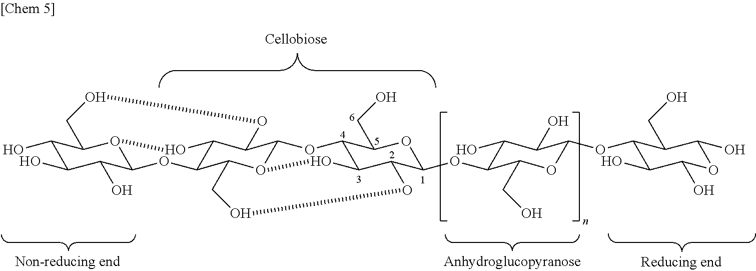

The hydroxyl groups of cellulose can react partially or completely with various chemical reagents to give cellulose derivatives having intrinsic properties. The cellulose derivatives can be anionic or non-ionic. Among these derivatives, cellulose ethers, cellulose esters and cellulose ether esters are distinguished.

Mention may be made, among the non-ionic cellulose ethers, of alkylcelluloses, such as methylcelluloses and ethylcelluloses; hydroxyalkylcelluloses, such as hydroxymethylcelluloses, hydroxyethylcelluloses and hydroxypropylcelluloses; and mixed hydroxyalkylalkylcelluloses, such as hydroxypropylmethylcelluloses, hydroxyethylmethylcelluloses, hydroxyethylethylcelluloses and hydroxybutylmethylcelluloses.

Mention may be made, among the anionic cellulose ethers, of carboxyalkylcelluloses and their salts. Mention may be made, by way of example, of carboxymethylcelluloses, carboxymethylmethylcelluloses and carboxymethylhydroxyethylcelluloses and their sodium salts.

Mention may also be made, among the cellulose derivatives, of:
celluloses modified by groups comprising at least one fatty chain, such as, for example, hydroxyethylcelluloses modified by groups comprising at least one fatty chain, such as alkyl, in particular C₈-C₂₂ alkyl, arylalkyl or alkylaryl groups, such as Natrosol® Plus Grade 330 CS (C₁₆ alkyls) sold by Ashland. and
celluloses modified by polyalkylene glycol alkylphenyl ether groups, such as the product Amercell Polymer HM-1500 (nonylphenol polyethylene glycol (15) ether) sold by Amerchol.

The cellulose esters include cellulose inorganic esters (cellulose nitrates, sulfates, phosphates, and the like), cellulose organic esters (cellulose monoacetates, triacetates, amidopropionates, acetate butyrates, acetate propionates and acetate trimellitates, and the like), and mixed cellulose organic/inorganic esters, such as cellulose acetate butyrate sulfates and cellulose acetate propionate sulfates. Mention may be made, among the cellulose ether esters, of hydroxypropylmethylcellulose phthalates and ethylcellulose sulfates.

The cellulose-based compounds of the invention can be chosen from unsubstituted celluloses and substituted celluloses.

The celluloses and derivatives are represented, for example, by the products sold under the names Avicel® (microcrystalline cellulose, MCC) by FMC Biopolymers, under the name Cekol® (carboxymethylcellulose) by Noviant (CP-Kelco), under the name Akucell AF® (sodium carboxymethylcellulose) by Akzo Nobel, under the name Aquasorb A® 500 by Ashland (sodium carboxymethylcellulose), under the names Methocel™ (cellulose ethers) and Ethocel™ (ethylcellulose) by Dow, under the names Aqualon® (carboxymethylcellulose and sodium carboxymethylcellulose), Benecel® (methylcellulose), Blanose™ (carboxymethylcellulose), Culminal® (methylcellulose, hydroxypropyl methylcellulose), Klucel® (hydroxypropylcellulose) and Polysurf® (cetyl hydroxyethylcellulose), under the name Natrosol® Plus 330 CS by Ashland (alkyl (C_{14/16}) hydroxyethyl cellulose), under the name Natrosol® 250 HHR CS by Ashland (hydroxyethyl cellulose (MW: 1 300 000)) and Natrosol® CS (hydroxyethylcellulose) by Hercules Aqualon.

According to a specific embodiment, the composition of the invention is employed in the presence of hydroxyethyl cellulose, in particular sold under the trade name Natrosol® 250 HHR CS by Ashland, or also of sodium carboxymethylcellulose, in particular sold under the trade name Aquasorb A® 500 by Ashland.

The cellulose or its derivatives can be employed in a proportion of 0.1% to 5% by weight of dry matter with respect to the total weight of the composition, in particular from 0.5% to 3% by weight, preferably between 1% and 2.5% by weight, with respect to the total weight of the composition.

According again to a specific embodiment, the hydrophilic gelling agent can be chosen from (iii) at least one carboxyvinyl polymer.

### Carboxyvinyl Polymers

The modified or unmodified carboxyvinyl polymers can be copolymers resulting from the polymerization of at least one monomer (a) chosen from α,β-ethylenically unsaturated carboxylic acids or their esters, with at least one ethylenically unsaturated monomer (b) comprising a hydrophobic group.

The term “copolymers” is understood to mean both copolymers obtained from two kinds of monomer and those obtained from more than two kinds of monomer, such as terpolymers obtained from three kinds of monomer.

Their chemical structure more particularly comprises at least one hydrophilic unit and at least one hydrophobic unit. The term “hydrophobic group” or “hydrophobic unit” is understood to mean a radical having a saturated or unsaturated and linear or branched hydrocarbon chain, comprising at least 8 carbon atoms, preferably from 10 to 30 carbon atoms, in particular from 12 to 30 carbon atoms and more preferentially from 18 to 30 carbon atoms.

Preferably, these copolymers are chosen from copolymers resulting from the polymerization:
of at least one monomer of following formula (1):

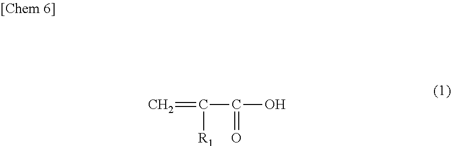

in which R₁ denotes H or CH₃ or C₂H₅, that is to say acrylic acid, methacrylic acid or ethacrylic acid monomers, and
of at least one monomer corresponding to the monomer of following formula (2):

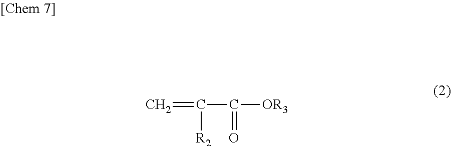

in which R₂ denotes H or CH₃ or C₂H₅ (that is to say, acrylate, methacrylate or ethacrylate units) and preferably H (acrylate units) or CH₃ (methacrylate units), and R₃ is a hydrogen atom.

According to a preferred embodiment, these polymers are crosslinked.

Use will more particularly be made, among copolymers of this type, of polymers resulting from the polymerization of a mixture of monomers comprising:
essentially acrylic acid,
a compound of formula (2) described above in which R₂ denotes H or CH₃, and R₃ is a hydrogen atom,
a crosslinking agent, which is a well-known copolymerizable unsaturated polyethylenic monomer, such as diallyl phthalate, allyl (meth)acrylate, divinylbenzene, (poly)ethylene glycol dimethacrylate and methylenebisacrylamide.

According to a specific embodiment of the invention, the carboxyvinyl polymers employed in the context of the present invention are unmodified. They are in particular homopolymers.

Mention may also be made, among the carboxyvinyl polymers, of sodium polyacrylates, such as those sold under the name Cosmedia SP® containing 90% dry matter and 10% water, or Cosmedia SPL® as an inverse emulsion containing approximately 60% dry matter, an oil (hydrogenated polydecene) and a surfactant (PPG-5 Laureth-5), both sold by BASF.

Mention may also be made of partially neutralized sodium polyacrylates which are in the form of an inverse emulsion comprising at least one polar oil, for example that sold under the name Luvigel® EM by BASF.

The modified or unmodified carboxyvinyl polymers can also be chosen from crosslinked (meth)acrylic acid homopolymers.

Within the meaning of the present patent application, the term “(meth)acrylic” is understood to mean “acrylic or methacrylic”.

Mention may be made, by way of example, of those sold by Lubrizol under the names Carbopol 910, 934, 940, 941, 934 P, 980, 981, 2984 or 5984, or by 3V-Sigma under the name Synthalen® K, Synthalen® L or Synthalen® M.

According to another specific embodiment of the invention, mention may be made, among the carboxyvinyl polymers, of crosslinked acrylic acid homopolymers. Mention may in particular be made, among the carboxyvinyl polymers which can more particularly be employed in the context of the present invention, of Carbopol (CTFA name: carbomer) sold by Lubrizol.

Mention may be made, among the homopolymers of this type, of those crosslinked by an allyl ether of alcohol of the sugar series, such as, for example, the products sold under the names Carbopol 980, 981, 954, 2984 and 5984 by Lubrizol or the products sold under the names Synthalen® M and Synthalen® K by 3V.

Such acrylic homopolymers can be present in the composition in a particulate or non-particulate form. When they are provided in a particulate form, their mean size in the hydrated state is preferably less than or equal to 10 μm and more preferentially still less than or equal to 5 μm. Their mean size in the dry or non-hydrated state is preferably less than or equal to 2 μm, preferably less than or equal to 1 μm.

Preferably, the acrylic acid homopolymer is present in non-particulate form.

Preferably, use is made of an at least partially neutralized acrylic acid homopolymer. The homopolymer used according to the invention can be chosen in particular from sodium polyacrylates and potassium polyacrylates. Sodium polyacrylate is preferably used.

Mention is made, as regards these acrylic polymers already neutralized before they are employed, for example, of:
sodium polyacrylates, such as those sold under the name Cosmedia SP® containing 90% dry matter and 10% water, or Cosmedia SPL® as an inverse emulsion containing approximately 60% dry active material, an oil (hydrogenated polydecene) and a surfactant (PPG-5 laureth-5), both sold by BASF;
partially neutralized sodium polyacrylates, which are in particular in the form of an inverse emulsion comprising at least one polar oil, for example that sold under the name Luvigel® EM by BASF; and
their mixtures.

The carboxyvinyl polymers, which are more particularly unmodified, can be present in a proportion of 0.1% to 5% by weight of dry matter, with respect to the weight of the aqueous phase, in particular from 0.3% to 3% by weight, preferably between 0.5% and 2% by weight, with respect to the weight of the aqueous phase.

The abovementioned gelling agents can also be present in the composition according to the present invention as a mixture.

According to a specific embodiment of the invention, the hydrophilic gelling agent is chosen from crosslinked ammonium acrylamido-2-methylpropanesulfonate polymers, crosslinked acrylamide/sodium acrylamido-2-methylpropanesulfonate copolymers, copolymers of AMPS® and of hydroxyethyl acrylate, xanthan gum, carboxymethylcelluloses or one of their salts, hydroxyethylcelluloses modified by C₈-C₂₂ alkyl groups, sodium polyacrylates, crosslinked (meth)acrylic acid homopolymers, and their mixtures.

Additional gelling agents can also be present in the composition according to the present invention. It is a matter of routine operations for a person skilled in the art to adjust the nature and the amount of such additional gelling agents present in the compositions in accordance with the invention so that the cosmetic properties desired for these are not thereby affected.

### Aqueous Phase

The aqueous phase comprises water and optionally a water-soluble solvent.

According to the present invention, the term “water-soluble solvent” is understood to denote a compound which is liquid at ambient temperature and water-miscible (miscibility in water of greater than 50% by weight at 25° C. and atmospheric pressure).

The water-soluble solvents which can be used in the composition of the invention can in addition be volatile.

Mention may in particular be made, among the water-soluble solvents which can be used in the composition according to the invention, of lower monoalcohols having from 1 to 5 carbon atoms, such as ethanol and isopropanol, glycols having from 2 to 8 carbon atoms, such as ethylene glycol, hexylene glycol, propylene glycol, 1,3-butylene glycol and dipropylene glycol, C₃ and C₄ ketones and C₂-C₄ aldehydes.

According to an alternative embodiment, the aqueous phase of a composition according to the invention can comprise at least one C₂-C₃₂ polyol.

Within the meaning of the present invention, the term “polyol” should be understood as meaning any organic molecule comprising at least two free hydroxyl groups.

Preferably, a polyol in accordance with the present invention is present in liquid form at ambient temperature.

A polyol suitable for the invention can be a compound of saturated or unsaturated and linear, branched or cyclic alkyl type carrying, on the alkyl chain, at least two —OH functional groups, in particular at least three —OH functional groups and more particularly at least four —OH functional groups.

The polyols suitable for the formulation of a composition according to the present invention are in particular those exhibiting in particular from 2 to 32 carbon atoms, preferably from 3 to 16 carbon atoms.

The polyol can, for example, be chosen from ethylene glycol, pentaerythritol, trimethylolpropane, propylene glycol, dipropylene glycol, 1,3-propanediol, butylene glycol, isoprene glycol, pentylene glycol, hexylene glycol, caprylyl glycol, glycerol, polyglycerols, such as glycerol oligomers, for instance diglycerol, polyethylene glycols, and their mixtures. According to a preferred embodiment of the invention, said polyol is chosen from ethylene glycol, pentaerythritol, trimethylolpropane, propylene glycol, dipropylene glycol, caprylyl glycol, glycerol, polyglycerols, polyethylene glycols, and their mixtures.

According to a preferred embodiment, the composition of the invention comprises at least glycerol.

Preferably, a composition according to the invention comprises from 2% to 20% by weight of glycerol, in particular from 3% to 15% by weight and more preferentially from 4% to 10% by weight of glycerol, with respect to the total weight of the composition.

Preferably, the aqueous phase is present in a composition according to the invention in a content ranging from 85% to 100% by weight, preferably from 90% to 100% by weight and more preferentially from 98% to 100% by weight, with respect to the total weight of said composition.

When the composition is in the form of an aqueous gel, it can advantageously be provided in the form of a threshold aqueous gel.

In the context of the present invention, the term “threshold aqueous gel” or “threshold gel” is understood to mean an aqueous gel, the flow of which only takes place above a certain value of stress to be applied to said aqueous gel, referred to as yield point, yield value, yield stress, critical stress or threshold stress. In other words, said aqueous gel reacts as a solid below this threshold stress (it does not deform or reacts elastically, returning to the initial state after the stress). However, above this threshold stress, it behaves as a fluid gel and flows.

### Fatty Phase

The compositions according to the invention can contain at least one water-immiscible liquid organic phase, known as fatty phase. Such a fatty phase can thus be included in a content ranging up to 10% by weight, with respect to the total weight of the composition.

When it is present, it generally comprises one or more hydrophobic compounds which render said phase water-immiscible. This phase is liquid (in the absence of structuring agent) at ambient temperature (20-25° C.). Preferentially, the water-immiscible liquid organic phase according to the invention comprises at least one volatile oil and/or one non-volatile oil and optionally at least one structuring agent.

In the context of the present invention, the term “oil” means a fatty substance which is liquid at ambient temperature (25° C.) and atmospheric pressure (760 mml-Ig, i.e. 1.05×10⁵ Pa).

The oil can be chosen from any physiologically acceptable oil and particularly cosmetically acceptable oil, in particular mineral, animal, plant or synthetic oils; in particular, hydrocarbons oils, which are volatile or non-volatile, and/or silicone and/or fluorinated oils, and their mixtures.

Mention may be made, as examples of oils which can be used in the invention, of:
i) hydrocarbon vegetable oils, such as liquid triglycerides of fatty acids having 4 to 24 carbon atoms, such as caprylic/capric acid triglycerides, such as those sold by Stearineries Dubois or those sold under the names Miglyol 810, 812 and 818 by Dynamit Nobel, or jojoba oil;
ii) linear or branched hydrocarbons of mineral or synthetic origin, such as liquid paraffins and their derivatives, liquid petroleum, polydecenes, polybutenes, hydrogenated polyisobutene, such as Parleam, or squalane;
iii) synthetic ethers having from 10 to 40 carbon atoms;
iv) all synthetic esters, such as isononyl isononanoate, isopropyl myristate, isopropyl palmitate, C₁₂ to C₁₅ alkyl benzoate (list to be extended), silicone oils, such as non-volatile polydimethylsiloxanes (PDMSs) which are linear (dimethicones) or cyclic (cyclomethicones);
v) lipophilic derivatives of amino acids, such as isopropyl lauroyl sarcosinate (INCI name: Isopropyl Lauroyl Sarcosinate), sold under the name Eldew SL 205 by Ajinomoto; and
vi) their mixtures.

### Additives

A composition according to the invention can additionally comprise at least one additive chosen from the usual adjuvants in the cosmetic field, such as preservatives, fragrances, colorants, polar additives or chelating agents.

Of course, a person skilled in the art will take care to choose this or these optional additional compound(s) and/or their amount such that the advantageous properties of a composition according to the invention are not, or not substantially, detrimentally affected by the envisioned addition.

The composition according to the invention can additionally comprise other active constituents, such as desquamating agents, depigmenting or propigmenting agents, anti-glycation, anti-inflammatory or soothing agents, healing agents, moisturizing agents, humectants, agents for combatting pollution, anti-ageing agents, agents which stimulate the synthesis of dermal or epidermal macromolecules and/or which prevent their decomposition, agents which stimulate the proliferation of fibroblasts and/or keratinocytes or which stimulate the differentiation of keratinocytes, dermo-relaxing agents, tightening agents, agents which act on the microcirculation, agents which act on the energy metabolism of cells, mattifying agents, UV screening agents, odour absorbers or mixtures of these.

According to a preferred embodiment, a composition according to the invention also comprises at least one additional cosmetic active agent, in particular at least two additional cosmetic active agents and preferably at least three additional cosmetic active agents, other than the short-chain fatty acid salt, in particular other than sodium propionate.

In particular, the additional cosmetic active agent can be at least one hydrophilic active agent. “Hydrophilic active agent” is understood to mean a water-soluble or water-dispersible active agent capable of forming hydrogen bonds.

Mention may be made, as hydrophilic active agents, for example, of moisturizing agents, depigmenting agents, desquamating agents, humectants, anti-ageing agents, mattifying agents, healing agents, antibacterial agents and their mixtures.

The additional hydrophilic active agent(s) can in particular be chosen from:
vitamins and their derivatives, in particular niacinamide (vitamin B3);
humectants, such as urea, hydroxyureas, glycerol, polyglycerols, glyceryl glucoside, diglyceryl glucoside, polyglyceryl glucosides and xylityl glucoside, and in particular glycerol;
C-glycoside compounds, such as, for example, the C-glycoside compounds described in the document WO 02/051828; preferably, a C-glycoside suitable for the invention is C-β-D-xylopyranoside-2-hydroxypropane, the INCI name of which is Hydroxypropyl Tetrahydropyrantriol, in particular sold under the name Mexoryl SBB® or Mexoryl SCN® by Chimex;
antioxidant compounds;
in addition particularly anti-ageing active agents, such as hyaluronic acid compounds, and in particular sodium hyaluronate, salicylic acid compounds and especially 5-(n-octanoyl)salicylic acid (capryloylsalicylic acid), adenosine, C-β-D-xylopyranoside-2-hydroxypropane and the sodium salt of (3-hydroxy-2-pentylcyclopentyl)acetic acid; and
their mixtures.

According to another specific embodiment, the composition according to the invention, in addition to its abilities to promote sebum secretion, also makes possible moisturization of the skin.

### Purpose of the Composition

A composition according to the invention can be provided in the form of a cosmetic composition for caring for keratin materials, in particular the skin, preferably of a cosmetic composition for caring for the body or the face, preferably the face.

These compositions can constitute protecting, treating or caring gels for the face, for the hands or for the body, for example gels for daytime or night-time application, or protective or care body gels.

In particular, a composition of the invention can be provided in the form of an anti-ageing care composition for the skin of the body or of the face, in particular of the face.

Thus, the invention also relates to the use of a composition according to the invention for caring for keratin materials, in particular the skin, preferably for caring for in particular the skin of the body and/or of the face.

The invention also relates to a cosmetic process for caring for keratin materials, in particular the skin, comprising at least one stage of application to said keratin materials of a composition as defined above.

In particular, a composition according to the invention can be employed with the aim of combatting dry skin.

### Dry Skin

Dry skin manifests itself essentially in a sensation of discomfort, such as tautness and/or tightness. Said dry skin is also rough to the touch and/or appears to be covered with squamae. When the skin is slightly dry, the squamae are abundant but barely visible to the naked eye. They become less and less numerous, but increasingly visible to the naked eye, when this disorder worsens.

The cause of the dryness of the skin can be of constitutional or acquired type.

In a specific embodiment, the composition according to the invention is used for treating and/or preventing non-pathological constitutional dry skin or non-pathological acquired dry skin.

In the case of acquired dry skin, the involvement of external parameters, such as exposure to chemical agents, to inclement weather conditions or to the sun, or else certain therapeutic treatments (retinoids, for example), are determining factors. Under these external influences, the skin may then become momentarily and locally dry.

Non-pathological constitutional dry skin is dry skin, the severity of which can depend on the external factors already indicated. Included in this skin category, referred to as dry skin, are senile skin (characterized by a general decrease in skin metabolism with age), fragile skin (very sensitive to external factors and often accompanied by erythema and rosacea) and common xerosis (of probable genetic origin and manifesting itself mainly on the face, the limbs and the back of the hands).

The composition according to the invention thus proves to be particularly effective for preventing and/or treating dry skin, and more particularly acquired dry skin and/or constitutional dry skin.

Non-pathological acquired constitutional dry skin can be characterized by a deficiency in lipids constituting the hydro-lipid barrier and/or film, in particular an endogenous insufficiency of sebum production by the sebaceous glands.

As mentioned above, a sebum content of less than 100 g/cm², measured in the T zone of the face by the method described in FR 2 368 708, can be regarded as characteristic of hyposeborrhoeic dry skin.

A composition according to the invention thus proves to be particularly effective for preventing and/or treating hyposeborrhoeic dry skin.

In particular, a composition according to the invention can be employed for the purpose of combatting signs of skin ageing, in particular by promoting sebum production in aged skin, in particular of more than 60 years, indeed even of more than 65 years.

Thus, the present patent application also relates to the use of a composition according to the invention in order to combat signs of skin ageing.

### Aged Skin

The composition according to the invention proves to be particularly effective for preventing and/or treating aged skin, and more particularly hyposeborrhoeic aged skin. As mentioned above, premature ageing is known to be attributed to the absence or the insufficiency of sebum at the skin surface.

The term “aged skin” is understood to mean a general aesthetic state of the skin resulting from chronological ageing and/or from photoinduced ageing.

More particularly, the present invention is targeted at preventing and/or reducing and/or treating signs of skin ageing.

The term “signs of skin ageing” is understood to mean any modification of the external appearance of the skin due to ageing of chronological and/or photoinduced origin.

Mention may be made, by way of example of this modification considered in the invention, of a surface which is not very homogeneous and is less smooth, a thinned epidermis, wrinkles and fine lines, withered skin, a lack of elasticity and/or of tone of the skin, resulting in the appearance of flaccid and wrinkled skin.

In particular, the signs of skin ageing targeted by the invention are chosen from thinning of the skin, a loss of firmness, a loss of elasticity, a loss of density or a loss of tone of the skin, a detrimental change in the appearance of the surface of the skin, the appearance of a marked microrelief of the skin, the appearance of roughness, the formation and % or the presence of fine lines and/or wrinkles, a change in the radiance of the complexion, a withered appearance of the skin, sagging of the skin or withering of the skin.

Preferably, the signs of skin ageing targeted by the invention are chosen from thinning of the skin, the appearance of a marked microrelief of the skin, the formation and/or the presence of fine lines and/or wrinkles, slackening of the skin and withering of the skin.

More preferentially, the signs of skin ageing targeted by the invention are chosen from the appearance of a marked microrelief of the skin, the formation and/or the presence of fine lines and/or wrinkles, slackening of the skin and withering of the skin.

The composition can be applied to the skin by hand or using an applicator.

Throughout the description, including the claims, the expression “comprising a” should be understood as being synonymous with “comprising at least one”, unless otherwise specified. The expressions “between . . . and . . . ”, “comprises from . . . to . . . ”, “formed of . . . to . . . ” and “ranging from . . . to . . . ” should be understood as including the limits, unless otherwise specified.

The invention is illustrated in greater detail by the examples and figures presented below. Unless otherwise indicated, the amounts indicated are expressed as percentages by weight.

### Example

### Measurement and Evaluation Methods

### Stability Measurement

The stability of the compositions is evaluated at T=2 months, at 45° C. It is verified whether phase separation, release or a change in appearance is observed.

### Appearance

The appearance of the composition obtained is evaluated:
by observation of its behaviour from the viewpoint of its viscoelastic properties, and
by observation of its transparent or translucent appearance.

Different types of gels can thus be obtained:
“threshold gel”, as defined above,
runny gel, which, on the contrary, flows whatever the stress which is applied to it.

It should be noted that a runny gel exhibits a very low threshold; this is the reason why it flows easily, as soon as the bottle in which it is contained is tilted.

### Manufacturing Process for Limiting the Formation of Unpleasant Acid Odour

Whatever the polymeric or gelling system used, the cosmetic composition is preferably prepared in several stages:
1. Glycerol or any other hydrophilic starting material and the preservatives are introduced into water at approximately 60° C. and then the polymers are dispersed all together.
2. Before the introduction of the phase containing the sodium propionate (25° C.), the pH of the preceding phase(s) is adjusted to a pH of between 6.3 and 7.
3. The pH of the phase containing the sodium propionate is adjusted to a pH of between 6.3 and 7.
4. At 25° C., the phase comprising the sodium propionate is introduced into the mixture of the preceding phase(s) and homogenization is carried out. During the addition of the phase comprising the sodium propionate, an entirely acceptable fluidification of the preceding phase(s) is observed.
5. The pH is adjusted to between 6.3 and 7, if need be.

### Example 1: Preparation of an Aqueous Gel

An aqueous gel 1 according to the invention is prepared from the proportions by weight as described in detail in the table below. The values are expressed as percentages by weight, with respect to the total weight of the composition.

TABLE 1

| | INCI name | Aqueous gel 1 |
|---|---|---|
| Phase A | Aqua (water) | q.s. for 100 |
| | Hydroxyethyl acrylate/ | 2.4 |
| | sodium acryloyldimethyl | |
| | taurate copolymer (sold | |
| | under the trade name | |
| | Sepinov EMT 10 ® | |
| | by Seppic) | |
| Phase B | Aqua (water) | 12 |
| | Sodium propionate | 2.4 |
| | 10% Citric acid | 0.42 |
| | solution for adjusting | |
| | the pH of phase A to 6.3 | |
| Total | | 100 |
| pH | | pH = 6.3 |
| Results/ | | A threshold gel is obtained |
| appearance | | |
| Stability | | Threshold gel not detrimentally |
| 2 months | | affected/stability confirmed |
| 45° C. | | |

The polymer derived from ammonium polyacryloyldimethyltaurate employed in this example makes it possible to obtain appropriate and stable gelling.

### Example 2: Preparation of an Aqueous Gel

An aqueous gel 2 according to the invention is prepared from the proportions by weight as described in detail in the table below. The values are expressed as percentages by weight, with respect to the total weight of the composition.

TABLE 2

| | INCI name | Aqueous gel 2 |
|---|---|---|
| Phase A | | 85.52 |
| | Polyacrylamide (and) 13- | 5.9 (2.4 as |
| | 14 isoparaffin (and) | active material) |
| | laureth-7 (sold under the | |
| | trade name Sepigel | |
| | 305 ® by Seppic) | |
| Phase B | Aqua (water) | 10.0 |
| | Sodium propionate | 2.00 |
| | Triethanolamine | q.s. pH 6.3 |
| Total | | 100 |
| pH | | pH = 6.3 |
| Results | | A threshold gel is obtained |
| Stability | | Threshold gel not detrimentally |
| 2 months | | affected/stability confirmed |
| 45° C. | | |

The polymer derived from polyacrylamide employed in this example makes it possible to obtain appropriate and stable gelling.

### Example 3: Preparation of an Aqueous Gel

An aqueous gel 3 according to the invention is prepared from the proportions by weight as described in detail in the table below. The values are expressed as percentages by weight, with respect to the total weight of the composition.

TABLE 3

| Phase | INCI name | Aqueous gel 3 |
|---|---|---|
| Phase A | Hydroxyethyl Acrylate/Sodium | 3.00 |
| | Acryloyldimethyl Taurate Copolymer | |
| | (sold under the trade name | |
| | Sepinov EMT 10 ® by Seppic) | |
| Phase B | Aqua (water) | q.s. for 100 |
| | Phenoxyethanol (sold under the | 0.50 |
| | trade name Sepicide LD ® | |
| | by Seppic) | |
| | Caprylyl glycol (sold under the | 0.50 |
| | trade name Dermosoft Octiol ® | |
| | by Dr Straetmans -Evonik) | |
| | Glycerol | 5.00 |
| Phase C1 | Sodium propionate | 2.00 |
| Phase C2 | 10% Citric acid in water | 0.586 |
| | q.s. pH 6.3 | |
| | Aqua (water) | 0.37 |
| Phase E | 50% Sodium hydroxide in water | Qs pH 6.3-6.7 |
| | Aqua (water) | |
| Total | | 100 |
| pH | | 6.3 |
| Appearance | | A threshold gel is |
| | | obtained which is |
| | | creamy; translucent |

The polymer Hydroxyethyl Acrylate/Sodium Acryloyldimethyl Taurate Copolymer employed in this example makes it possible to obtain appropriate gelling.

### Example 4: Preparation of an Aqueous Gel

An aqueous gel 4 according to the invention is prepared from the proportions by weight as described in detail in the table below. The values are expressed as percentages by weight, with respect to the total weight of the composition.

TABLE 4

| | INCI name | Aqueous gel 4 |
|---|---|---|
| Phase A | Aqua (water) | 85.52 |
| | Hydroxyethyl | 2.00 |
| | Acrylate/Sodium | |
| | Acryloyldimethyl Taurate | |
| | Copolymer (sold under the | |
| | trade name Sepinov | |
| | EMT 10 ® by Seppic) | |
| Phase B | Aqua (water) | 10.00 |
| | Sodium propionate | 2.00 |
| | Triethanolamine | q.s. pH 6.3 |
| Total | | 100 |
| pH | | pH = 6.3 |
| Results | | A threshold gel is obtained |
| Stability | | Threshold gel not detrimentally |
| 2 months | | affected/stability confirmed |
| 45° C. | | |

The polymer Hydroxyethyl Acrylate/Sodium Acryloyldimethyl Taurate Copolymer employed in this example makes it possible to obtain appropriate and stable gelling.

### Example 5: Preparation of an Aqueous Gel

An aqueous gel 5 according to the invention is prepared from the proportions by weight as described in detail in the table below. The values are expressed as percentages by weight, with respect to the total weight of the composition.

TABLE 5

| | Trade name | Aqueous gel 5 |
|---|---|---|
| Phase A | Aqua (water) | q.s. for 100 |
| | Xanthan (sold under the trade | 1.30 |
| | name Rhodicare CFT ® by PMC | |
| | OUVRIE) | |
| Phase B | Aqua (water) | 10.00 |
| | Sodium propionate | 2.00 |
| | Glycerol | 5.00 |
| | Phenoxyethanol (sold under the | 0.70 |
| | trade name Sepicide LD ® by | |
| | Seppic) | |
| | CAprylyl glycol (sold under the | 0.50 |
| | trade name Dermosoft Octiol ® | |
| | by Dr Straetmans - Evonik) | |
| Total | | 100 |
| pH | | 6.3 |
| Results | | A viscous threshold gel |
| | | is obtained; creamy |

The xanthan employed in this example makes it possible to obtain appropriate gelling.

### Example 6: Preparation of an Aqueous Gel

An aqueous gel 6 according to the invention is prepared from the proportions by weight as described in detail in the table below. The values are expressed as percentages by weight with respect to the total weight of the composition.

TABLE 6

| | | Comparative | |
|---|---|---|---|
| | | gel without | |
| | | sodium | |
| | INCI name | propionate | Aqueous gel 6 |
| Phase A | | 99.00 | 85.52 |
| | Sodium | 1.00 | 1.00 |
| | carboxymethyl | | |
| | cellulose (sold | | |
| | under the trade | | |
| | name Aquasorb | | |
| | A ® by Ashland) | | |
| Phase B | Aqua (water) | | 10.00 |
| | Sodium propionate | | 2.00 |
| | Citric acid | | 0.05 |
| | Triethanolamine | | 0.14 |
| | | | 0.50 |
| Total | | 100 | 100 |
| pH | | 7.3 | Ph = 6.3 |
| Results | | Cloudy | A transparent gel is |
| | | runny | obtained. The visual |
| | | gel | image obtained is better |
| | | | than for the gel devoid |
| | | | of sodium propionate |

The cellulose polymer employed in this example makes it possible to obtain appropriate gelling.

Two other cellulose polymers are used in the same gel:
(C_{14/16}) alkyl hydroxyethyl cellulose sold under the trade name Natrosol® Plus 330 CS by Ashland, and
Hydroxyethyl cellulose (MW: 1 300 000), sold under the trade name Natrosol® 250 HHR CS by Ashland—see Example 7 below.

The gels obtained are also satisfactory in terms of appearance and of stability at 2 months at 45° C.

### Example 7: Preparation of an Aqueous Gel

An aqueous gel 7 according to the invention is prepared from the proportions by weight as described in detail in the table below. The values are expressed as percentages by weight, with respect to the total weight of the composition.

TABLE 7

| | INCI name | Aqueous gel 7 |
|---|---|---|
| Phase A | Aqua (water) | q.s. for 100 |
| | Hydroxyethyl | 2.00 |
| | Cellulose (sold under | |
| | the trade name | |
| | Natrosol ® 250 HHR | |
| | by Ashland; (MW: 1 | |
| | 300 000) | |
| Phase B | Aqua (water) | 10.00 |
| | Sodium propionate | 3.00 |
| | Glycerol | 5.00 |
| | Phenoxyethanol (sold | 0.70 |
| | under the trade name | |
| | Sepicide LD ® by | |
| | Seppic) | |
| | Caprylyl glycol (sold | 0.50 |
| | under the trade name | |
| | Dermosoft Octiol ® | |
| | by Straetmans - Evonik) | |
| Total | | 100 |
| pH | | Ph = 7.5 |
| Results | | A perfectly transparent, |
| | | creamy gel is obtained |

The cellulose polymer employed in this example makes it possible to obtain appropriate gelling.

### Example 8: Preparation of a Composition Comprising a Mixture of Gelling Agents, of Serum Type

A serum 1 according to the invention is prepared from the proportions by weight as described in detail in the table below. The values are expressed as percentages by weight, with respect to the total weight of the composition.

TABLE 8

| | INCI name | Serum 1 |
|---|---|---|
| Phase A | Xanthan (sold under the trade | 0.30 |
| | name Rhodicare CFT ® by PMC | |
| | OUVRIE) | |
| | Ammonium | 0.20 |
| | Polyacryldimethyltauramide (sold | |
| | under the trade name Hostacerin | |
| | AMPS ® by Clariant) | |
| | Crosslinked polyacrylic acid(sold | 0.40 |
| | under the trade name Carbopol 980 | |
| | Polimer ® by Lubrizol) | |
| Phase B | Aqua (water) | q.s. for 100 |
| | Phenoxyethanol (sold under the | 0.50 |
| | trade name Sepicide LDR by | |
| | Seppic) | |
| | Caprylyl glycol (sold under the | 0.50 |
| | trade name Dermosoft Octiol ® by | |
| | Straetmans - Evonik) | |
| | Glycerol | 5.00 |
| Phase C | Aqua (water) | 0.93 |
| | Sodium hydroxide | 0.93 |
| Phase D | Aqua (water) | 10.00 |
| | Sodium propionate | 2.00 |
| Phase E | 10% Citric acid in water | 0.586 |
| | q.s. pH 6.3 | |
| | Sodium hydroxide | q.s. ph 6.3 to 6.7 |
| TOTAL | | 100 |
| pH | | 6.3 |
| Results | | A gel is |
| | | obtained |

The mixture of xanthan, of Ammonium Polyacryldimethyltauramide and of crosslinked acrylic copolymer employed in this example makes it possible to obtain appropriate gelling.

### Example 9: Preparation of an Aqueous Gel Comprising a Mixture of Gelling Agents

An aqueous gel 8 according to the invention is prepared from the proportions by weight as described in detail in the table below. The values are expressed as percentages by weight, with respect to the total weight of the composition.

TABLE 9

| Phase | INCI name | Aqueous gel 8 |
|---|---|---|
| Phase A | Ammonium | 1.50 |
| | Polyacryldimethyltauramide | |
| | (sold under the trade | |
| | name Hostacerin AMPS ® | |
| | by Clariant) | |
| | Sodium polyacrylate (sold | 0.50 |
| | under the trade name | |
| | Cosmedia SP ® by BASF) | |
| Phase B | Aqua (water) | q.s. for 100 |
| | Phenoxyethanol (sold | 0.50 |
| | under the trade name | |
| | Sepicide LD ® by Seppic) | |
| | Caprylyl glycol (sold under | 0.50 |
| | the trade name Dermosoft | |
| | Octiol ® by Straetmans - | |
| | Evonik) | |
| | Glycerol | 5.00 |
| Phase C | Aqua (water) | 0.19 |
| | Sodium hydroxide | 0.19 |
| Phase D | Aqua (water) | 10.00 |
| | Sodium propionate | 2.00 |
| | 10% Citric acid in water | 0.586 |
| | q.s. pH 6.3 | |
| | Aqua (water) | 0.372 |
| Phase E | 50% Sodium hydroxide | q.s. ph 6.3 to 6.7 |
| | in water | |
| | Aqua (water) | If necessary |
| TOTAL | | 100 |
| pH | | 6.3 |
| Results | | A threshold gel is obtained |
| | | which is creamy; translucent |

The mixture of Ammonium Polyacryldimethyltauramide, of Ammonium Polyacryldimethyltauramide and of Sodium polyacrylate employed in this example makes it possible to obtain appropriate gelling.

### Example 10: Preparation of an Emulsified Gel Comprising a Mixture of Gelling Agents, and a Cosmetic Oil

An emulsified gel 1 according to the invention is prepared from the proportions by weight as described in detail in the table below. The values are expressed as percentages by weight, with respect to the total weight of the composition.

TABLE 10

| Phase | INCI name | Emulsified gel 1 |
|---|---|---|
| Phase A | Ammonium | 1.50 |
| | Polyacryldimethyltauramide (sold | |
| | under the trade name Hostacerin | |
| | AMPS ® by Clariant) | |
| | Sodium polyacrylate (sold under | 0.50 |
| | the trade name Cosmedia SP ® | |
| | by BASF) | |
| Phase B | Aqua (water) | q.s. for 100 |
| | Phenoxyethanol (sold under the | 0.50 |
| | trade name Sepicide LD ® | |
| | by Seppic) | |
| | Caprylyl glycol (sold under the | 0.50 |
| | trade name Dermosoft Octiol ® by | |
| | Straetmans - Evonik) | |
| | Glycerol | 5.00 |
| Phase C | Aqua (water) | 0.19 |
| | Sodium hydroxide | 0.19 |
| Phase D | Aqua (water) | 10.00 |
| | Sodium propionate | 2.00 |
| | 10% Citric acid in water | 0.586 |
| | q.s. p.H. 6.3 | |
| | Aqua (water) | 0.372 |
| Phase F | 50% Sodium hydroxide in water | q.s. ph 6.3-7 |
| Phase G | Caprylic/capric trigyceride (sold | 5 |
| | under the trade name Triglycerides | |
| | C8C10 70/30 (DUB MCT 7030 ®) | |
| | by Dr Stearinerie Dubois) | |
| | Aqua (water) | If necessary |
| TOTAL | | 100 |
| pH | | 6.3 |
| Results | | A threshold gel is |
| | | obtained which is |
| | | creamy; translucent |

The mixture of Ammonium Polyacryldimethyltauramide, of Ammonium Polyacryldimethyltauramide and of sodium polyacrylate employed in this example makes it possible to obtain appropriate gelling and the oil dispersion.

### Example 11: Preparation of an Emulsified Gel Comprising a Mixture of Gelling Agents, and a Cosmetic Oil

An emulsified gel 2 according to the invention is prepared from the proportions by weight as described in detail in the table below. The values are expressed as percentages by weight, with respect to the total weight of the composition.

TABLE 11

| Phase | INCI name | Emulsified gel 2 |
|---|---|---|
| Phase A | Hydroxyethyl | 3.00 |
| | Acrylate/Sodium | |
| | Acryloyldimethyl | |
| | Taurate Copolymer | |
| | (sold under the trade | |
| | name Spinov 10 ® by | |
| | Seppic) | |
| Phase B | Aqua (water) | q.s. for 100 |
| | Phenoxyethanol | 0.50 |
| | (sold under the trade | |
| | name Sepicide LD ® | |
| | by Seppic) | |
| | Caprylyl glycol (sold | 0.50 |
| | under the trade name | |
| | Dermosoft Octiol ® | |
| | by Straetmans - | |
| | Evonik) | |
| | Glycerol | 5.00 |
| Phase C1 | Sodium propionate | 2.00 |
| Phase C2 | 10% Citric acid in | 0.586 |
| | water q.s. pH 6.3 | |
| | Aqua (water) | 0.37 |
| Phase E | 50% Sodium | q.s. ph 6.3-7 |
| | hydroxide in water | |
| Phase F | Caprylic/capric | 5 |
| | trigyceride (sold | |
| | under the trade name | |
| | Triglycerides C8C10 | |
| | 70/30 (DUB MCT | |
| | 7030 ®) by Dr | |
| | Stearinerie Dubois) | |
| TOTAL | | 100 |
| pH | | 6.3 |
| Results | | A threshold gel is obtained |
| | | which is creamy; translucent |

The polymer Hydroxyethyl Acrylate/Sodium Acryloyldimethyl Taurate Copolymer employed in this example makes it possible to obtain appropriate gelling and the oil dispersion.

### Counterexample 1

A composition is prepared with an acrylates/C₁₀₋₃₀ alkyl acrylate crosspolymer gelling agent, such as, for example, Ultrez 20® or Pemulen TR21® which are sold by Lubrizol. Their compositions are as follows:

| | |
|---|---|
| acrylates/C₁₀₋₃₀ alkyl acrylate crosspolymer | 90 |
| trideceth-6 | 5 |
| PEG-30 dipolyhydroxystearate | 5 |

TABLE 12

| | | | Gel with sodium |
|---|---|---|---|
| | Gel without sodium | | propionate |
| | propionate (INCI name) | | (INCI name) |
| Phase A | Aqua (water) | 97.95 | 85.42 |
| | Acrylates/C10/C30 | 1.00 | 1 |
| | alkyl acrylate | | |
| | crosspolymer (sold | | |
| | under the trade | | |
| | name Ultrez 20 ® | | |
| | by Lubrizol) | | |
| Phase B | Sodium propionate | 0 | 2.00 |
| | Aqua (water) | 10.00 | 10 |
| | Citric acid | 0 | 0.05 |
| | Triethanolamine | 1.05 | 1.53 |
| TOTAL | | 100 | 100 |
| pH | | 6.3 | 6.3 |
| Results | | A viscous | A viscous |
| | | threshold gel | threshold gel |
| | | is obtained | is obtained |
| | | which is | |
| | | transparent | |
| | | and “creamy” | |
| Stability | | The gel is not | The gel is |
| 2 months | | detrimentally | detrimentally |
| 45° C. | | affected | affected; it |
| | | | becomes very |
| | | | brittle |

Although being known to be resistant to salts, the hydrophobic modified acrylic polymer (modified by C₁₀₋₃₀ alkyl chains) is not compatible with an aqueous gel comprising sodium propionate being obtained.

1. A cosmetic composition, comprising:
between 0.5% and 4% by weight of short-chain fatty acid salt, comprising a saturated or unsaturated and straight or branched aliphatic chain containing 3 to 8 carbon atoms, preferably 3 to 5 carbon atoms, with respect to the total weight of the composition, and
at least 90% by weight of an aqueous phase, with respect to the total weight of the composition, gelled by a hydrophilic gelling agent chosen from:
(i) at least one polymer chosen from polyacrylamides and 2-acrylamido-2-methylpropanesulfonic acid polymers and copolymers,
(ii) at least one polysaccharide produced by microorganisms or isolated from algae,
(iii) at least cellulose or one of its derivatives,
(iv) at least one carboxyvinyl polymer, or
(v) mixtures thereof.

2. The composition according to claim 1, wherein the short-chain fatty acid salt is chosen from a salt of propionic acid, of butyric acid, of valeric acid or of one of their mixtures, and the salt chosen from calcium salts, sodium salts, magnesium salts and potassium salts.

3. The composition according to claim 1, wherein the short-chain fatty acid salt is present in a content of between 0.8% and 4% by weight.

4. The composition according to claim 1, wherein the short-chain fatty acid salt is present in a content of between 1% and 3% by weight.

5. The composition according to claim 1, wherein the hydrophilic gelling agent is chosen from crosslinked ammonium acrylamido-2-methylpropanesulfonate polymers, crosslinked acrylamide/sodium acrylamido-2-methylpropanesulfonate copolymers, copolymers of AMPS® and of hydroxyethyl acrylate, xanthan gum, carboxymethylcelluloses or one of their salts, hydroxyethylcelluloses modified by C₈-C₂₂ alkyl groups, sodium polyacrylates, crosslinked (meth)acrylic acid homopolymers, and mixtures thereof.

6. The composition according to claim 1, wherein the aqueous phase is gelled by 0.1% to 8% by weight of at least one hydrophilic gelling agent, with respect to the total weight of the aqueous phase.

7. The composition according to claim 1, wherein the pH of the cosmetic composition according to the present invention is between 5.8 and 7.2.

8. The composition according to claim 1, wherein the aqueous phase comprises water and optionally a water-soluble solvent.

9. The composition according to claim 1, wherein it is provided in the form of an aqueous gel, of a serum or of an emulsified gel.

10. The composition according to claim 1, further comprising at least one additional cosmetic active agent.

11. The composition according to claim 1, wherein the composition is for caring for keratin materials.

12. A cosmetic process for caring for keratin materials comprising at least one stage of application to said keratin materials of the cosmetic composition of claim 1.
